(19) United States
(12) Patent Application Publication
(10) Pub. No.: US 2011/0159091 A1
(43) Pub. Date: Jun. 30, 2011

(54) RAPIDLY DISPERSIBLE VAGINAL TABLET THAT PROVIDES A BIOADHESIVE GEL

(76) Inventors: Alan Stone, London (GB); Alexander Robert Neurath, New York, NY (US); Nathan Strick, Oceanside, NY (US)

(21) Appl. No.: 12/601,517

(22) PCT Filed: May 23, 2008
(86) PCT No.: PCT/US08/64737

§ 371 Date: Aug. 24, 2010

Related U.S. Application Data
(60) Provisional application No. 60/931,548, filed on May 24, 2007.

(51) Int. Cl. A61K 9/20; A61K 31/717; A61K 31/765; A61K 31/775; A61P 31/18; A61P 31/20; A61P 31/04; A61P 33/02; A61K 31/737; A61K 31/565; A61K 31/4174; A61K 31/675; A61P 15/16

(52) U.S. Cl. 424/464; 514/57; 424/78.18; 514/54; 514/182; 514/396; 514/81

(57) ABSTRACT

A tablet for insertion into a vagina including 0.01 to 500 mg of a vaginal medication, such as a microbicide, such as cellulose acetate 1,2-benzenedicarboxylate (CAP); 100 to 500 mg of mannitol powder; 50 to 300 mg of inert microcrystalline cellulose; 10 to 80 mg of hydroxypropyl methylcellulose; 50 to 250 mg of glycerol and optionally 2 to 4 mg of at least one preservative which protects against microbicidal contamination and discourages the growth of yeast in the vagina. The tablet which includes CAP as the vaginal medication is vaginally administered before coitus in methods for preventing the sexual transmission of HIV-1, HIV-2, herpesvirus, or an infection caused by Neisseria gonorrhoeae, Chlamydia trachomatis, Trichomonas vaginalis, Haemophilus ducreyi or Treponema pallidum. The tablet which includes CAP as the vaginal medication is vaginally administered to prevent or treat bacterial vaginosis.

### CROSS REFERENCE TO RELATED APPLICATIONS

This application claims the benefit of priority under 35 USC 119(e) for U.S. provisional application Ser. No. 60/931,548 filed May 24, 2007, the entire contents of which are incorporated by reference herein.

### GOVERNMENT RIGHTS

This invention was made with United States government support under Grant Nos. U19 HD048957 and U19 AI076964 from the National Institute of Health. The United States government has certain rights in this invention.

### FIELD OF THE INVENTION

The present invention is directed to a tablet for insertion into a vagina, which disintegrates rapidly in the limited volume of fluid generally present in the vagina and rapidly forms a bioadhesive gel. More particularly, the present invention concerns a tablet for insertion into a vagina, wherein the tablet contains cellulose acetate 1,2-benzenedicarboxylate (“CAP”) as a microbicide.

### BACKGROUND OF THE INVENTION

The human immunodeficiency virus (HIV-1) pandemic has been driven primarily by the sexual transmission of the virus and facilitated by prior infections with other sexually transmitted disease (“STD”) pathogens. STDs of bacterial origin are a very common worldwide cause of illness and have significant health, social and economic consequences. They can lead to long-term, serious complications and consequences. The estimated annual worldwide incidence of the four major curable STDs, syphilis, gonorrhea, Chlamydia and trichomoniasis, is about 333 million. Another treatable STD, chancroid, caused by Haemophilus ducreyi, is common in developing countries in Africa, Asia and Latin America, where its incidence exceeds that of syphilis. Reported studies indicate that at least 20% of the United States population is infected with herpesvirus type 2 (HSV-2), which is predominantly transmitted sexually. The prevalence of HSV-2 is even higher in developing countries. Based on current statistics, it is expected that per year about 15 million people in the United States will acquire a STD, making the incidence of STDs in the United States the highest in the industrialized world. Currently, at least 66 million people (more than 1 in 3 adults age 15 to 65) in the United States are living with at least one STD.

The urgent need to prevent the transmission of STDs has become evident by the HIV-1/AIDS epidemic that has resulted so far in the infection of approximately 60 million people and in approximately 20 million deaths. AIDS is now the single leading infectious disease killer in the world. The observation that viral and non-viral STDs facilitate HIV-1 infection, emphasize the pressing need for preventive approaches against transmission of HIV-1 and other STDs. Such approaches include the use of chemical barrier methods (such as topical microbicides).

The transmission of HIV by heterosexual sex poses an especially severe problem for women. It is estimated that approximately 90% of HIV infections are acquired via heterosexual intercourse.

The utilization of condoms provides a substantial degree of protection against transmission of HIV, herpesvirus and other STD infections during sexual intercourse, but a difficulty arises when condoms are not employed. Moreover, the use of condoms appears to be a culturally and socially unacceptable practice in many countries.

Men can protect themselves and their partners from sexually transmitted HIV, herpesvirus and other STD infections if they use condoms. Women very often cannot persuade their male sex partners to use a condom. The female condom, which is just becoming available, is expensive and infrequently used.

Even if a woman maintains a monogamous sexual relationship, there is no guarantee of safety, for if a woman's male partner becomes infected, he can pass the virus to her and vice versa. As more women are infected, they are likely to transmit HIV-1 to their offspring.

As an alternative to gels for the delivery of vaginal medications, including microbicides, to prevent or treat STDs, it has been desired to develop solid dosage formulations (tablets, pessaries or suppositories) that can be inserted into the vagina using either the fingers or an appropriate applicator. With vaginal pessaries for the treatment of existing conditions, such as vaginal thrush (yeast infection; candidiasis), it is permissible for the medication to be released slowly over time. However, if a microbicide tablet is to be effective in preventing the transmission of HIV and other STD pathogens during coitus, the tablet must disintegrate rapidly in the presence of minimal volumes of vaginal fluids. The tablet must also quickly form a smooth, non-gritty, bioadhesive gel, and this gel must be readily miscible with biological fluids, i.e., the woman's own secretions or the man's semen. It is also desirable that the gel is able to maintain the vaginal contents at an acid pH, even after the entry of semen, which is alkaline. This is because vaginal acidity is inhospitable to HIV and contributes to the vagina's ability to resist colonization by pathogenic organisms, including HIV.

Hence, a solid dosage formulation such as a tablet for insertion into the vagina must contain specific components to meet the aforesaid critical requirements. The gel formed by such solid dosage formulations should preferably have desirable bioadhesive properties (to coat the vaginal epithelium and to prevent the gel from leaking from the vagina), have desirable tactile “feel” (including viscosity, smoothness and lack of grittiness), be miscible with biological fluids, be stable over time and have the requisite biological activity.

Rapidly dispersible oral tablets are well-known and widely used. The shared property between oral tablets and vaginal tablets is rapid dispersion, indicating the need for shared ingredients. As noted above, vaginal tablets must have additional properties such as the following:
(1) no grittiness (which puts a severe constraint on the availability and selection of ingredients);
(2) bioadhesive properties (these properties are undesirable for oral tablets which should be easily swallowed and should not stick to the tongue, throat, etc.);
(3) appropriate viscosity/rheological properties supporting vaginal residence, but allowing miscibility with physiological fluids; and
(4) increased requirements for safety.

The fastest way to introduce topical microbicides into practice would be the application of drugs or pharmaceutical ingredients that are already approved for other uses. Such microbicides should: (a) preferably not be spread systemically after topical application; (b) be inexpensive; (c) be produced from widely available resources; (d) have a broad specificity resulting in preventing the transmission of several STDs; (e) have a well-established, documented safety record; and (f) inactivate the infectivity of the respective STD pathogens, as implied in the word “microbicide.” CAP meets these criteria.

CAP is a short name for “cellulose acetate phthalate” (now more correctly referred to as cellulose acetate 1,2-benzenedicarboxylate) CAP is inexpensive and readily available in bulk quantities (it is manufactured as a widely used coating for oral tablets) and has a well-documented safety record established from oral use of CAP in humans, and from the application of large daily doses of CAP orally in dogs for a period of one year. The safety of undiluted CAP formulations has been demonstrated in in vitro and in ex vivo assays. Furthermore, the safety of undiluted CAP formulations applied vaginally was demonstrated in three model systems: (1) the rabbit vaginal irritation test, conducted according to FDA-approved conditions; (2) in extensive formal safety studies in pig-tailed macaques; and (3) in macaques as part of efficacy evaluations.

In the light of studies to date, experts in the field readily acknowledge that CAP could be potentially safe and effective (provided that efficacy is demonstrated in phase III human efficacy trials) as a microbicide.

The safety of soluble and micronized forms of CAP has been heretofore established in detail as shown in published papers and safety data from the manufacturers of CAP and the micronized form of CAP (Aquateric®), respectively (Eastman Chemical Company, Kingsport, Tenn.; FMC Corporation, Philadelphia, Pa.).

Single and Repeat Dose Toxicity and Carcinogenicity Studies

Single Dose Toxicity Studies

CAP can be found in the Inactive Ingredient Guide, where it is defined as an approved drug excipient currently marketed for human use for oral dosage forms. CAP safety has been extensively studied and it has been shown to be free of adverse effects (Neurath A R, Strick N, Li Y Y, Lin K, Jiang S, “Design of a ‘microbicide’ for prevention of sexually transmitted diseases using ‘inactive’ pharmaceutical excipients,” Biologicals, 17:11-21 (199-9)).

FMC Corporation (Philadelphia, Pa.) (U.S. Pharmacopeial Convention, Inc. The U.S. Pharmacopeia; pp. 780-781, (2000)) has performed extensive toxicity testing on micronized form of CAP, i.e., Aquateric® (containing 66-73 wt. % micronized CAP, a polyoxyethylene-polyoxypropylene block copolymer and distilled acetylated monoglycerides). The following Tables 1 and 2 contain toxicological information on Aquateric® from the FMC Corporation Material Safety Data Sheet.

TABLE 1
Toxicological Information for Aquateric ®

| Type of Study | Results | Animal Mode |
|---|---|---|
| Eye Irritation | Non-irritating | Rabbits |
| Dermal Irritation | Non-irritating | Rabbits |
| Dermal Sensitization | Non-sensitizing | Guinea Pig |
| Skin Absorption | Dermal LD₅₀ > 2,000 mg/kg | Rabbit |
| Inhalation | LC₅₀ > 5.21 mg/L/4 hr | Rat |
| | (maximum attainable | |
| | concentration, no mortalities) | |
| Ingestion | Oral LD₅₀ > 5,000 mg/kg | Rat |

Repeat Dose Toxicity and Carcinogenicity Studies

Rat (Oral Study)

Kotkoskie et al. (Kotkoskie L A, Freeman C, Palmieri M A, “Subchronic toxicity and developmental toxicity studies in rats with Aquateric® aqueous enteric coating,” Internat. J. Toxicology, 18:109-116 (1999)) examined the subchronic toxicity of Aquateric® (containing 66-73% micronized Cellulose Acetate 1,2-Benzenedicarboxylate (Cellulose Acetate Phthalate: CAP)) in four groups of twenty male and twenty female rats fed 0, 5,000, 25,000, or 50,000 ppm of Aquateric® daily for 90 days. No deaths occurred during the study and no treatment-related, clinical signs were noted. Clinical chemistry investigations yielded no toxicologically significant findings and all incidental findings were within physiologically acceptable historical reference ranges. There were likewise no treatment-related effects on organ weights or organ- to body-weight ratios. Based upon these study results, the No-Observed-Adverse-Effect-Level [NOAEL; greatest concentration of amount of a substance found by experiment or observation which causes no detectable adverse alteration of morphology, functional capacity, growth, development or life-span of the

TABLE 2
Summary of CAP Single Dose Toxicity Studies (from FMC Corporation)

| Species | | Group | Test | | | |
|---|---|---|---|---|---|---|
| (Strain) | Route | Size | Article | Dose/Duration | Results | Reference |
| Rat | Oral | 5 M + 5 F | 66-73 wt. % | 5,000 mg/kg/ | There were no deaths during the 14-day | FMC |
| (Sprague- | | | CAP in | single oral dose | observation period. The only clinical signs | Corporation |
| Dawley) | | | Aquateric ® | | observed were oral discharge in one female rat | study #: I83-796 |
| | | | CD-910 | | and diarrhea in one male rat. Aquateric ® CD-910 | |
| | | | | | is classified as practically non-toxic (the LD₅₀ is | |
| | | | | | greater than 5,000 mg/kg | |
| Rat | Inhalation | 5 M + 5 F | 66-73 wt. % | gravimetric | During the 15-day observation period, there were | FMC |
| (not | | | CAP in | concentration of | no deaths and no signs of toxicity among the test | Corporation |
| specified) | | | Aquateric ® | 5.21 mg/l/4 h | animals. Irregular breathing and poor coat quality | study #: I83-800 |
| | | | CD-910 | | were observed among the test animals during the | |
| | | | | | exposure. No gross lesions were found at | |
| | | | | | necropsy. Aquateric ® CD-910 is considered | |
| | | | | | practically non-toxic (the LD₅₀ is greater than 5.21 mg/l). | |
| Guinea Pig | Topical | 10 M + 10 F | 66-73 wt. % | 0.30 g (solid)/6 h | Aquateric ® CD-910 is judged to be non-sensitizing | FMC |
| (Hartley) | | | CAP in | three induction | when topically applied to Hartley guinea pigs. No | Corporation |
| | | | Aquateric ® | treatments one | responses were noted among test animals | study #: I92- |
| | | | CD-910 | week apart | following either the induction or challenge | 1266 |
| | | | | | application. No irritation was noted among any of | |
| | | | | | the challenge control guinea pigs during challenge. | |
| | | | | | Animals in the positive control group exhibited | |
| | | | | | definite sensitizing reactions following the | |
| | | | | | challenge application. | |
| Rabbit | Topical | 5 M + 5 F | 66-73 wt. % | 2,000 mg/kg/24 h | Aquateric ® CD-910 is classified as practically non- | FMC |
| (New | | | CAP in | | toxic (the LD₅₀ is greater than 2000 mg/kg). There | Corporation |
| Zealand | | | Aquateric ® | | were no deaths during the 14-day observation | study #: I83-797 |
| White) | | | CD-910 | | period. No skin irritation was observed in any | |
| | | | | | rabbit during the study. One rabbit had nasal | |
| | | | | | discharge and one rabbit had lacrimation. All but | |
| | | | | | two rabbits lost weight. | |
| Rabbit | Topical | 6 animals | 66-73 wt. % | 0.50 g/4 h | Aquateric ® CD-910 is judged to be non-corrosive | FMC |
| (New | | | CAP in | | and non-irritating to intact skin when applied | Corporation |
| Zealand | | | Aquateric ® | | topically to New Zealand White rabbits. No dermal | study #: I83-799 |
| White) | | | CD-910 | | irritation or corrosion was observed on any animal | |
| | | | | | during any of the scoring intervals. | |
| Rabbit | Ocular | 3 animals | 66-73 wt. % | 0.10 gm in the | Aquateric ® CD-910 is considered non-irritating to | FMC |
| (New | | in washed | CAP in | right eye/ | both washed and unwashed eyes. One hour after | Corporation |
| Zealand | | group | Aquateric ® | washed group: | dosing, slight conjunctivitis was observed in 4 of 6 | study #: I83-798 |
| White) | | 6 animals | CD-910 | eyes washed | unwashed eyes and 1 of 3 washed eyes. At 24 | |
| | | in | | after 20-30 sec. | hours, one of the unwashed eyes had slight | |
| | | unwashed | | of treatment | chemosis. All other eyes had returned to normal. | |
| | | group) | | 0.10 gm in the | | |
| | | | | right eye/ | | |
| | | | | unwashed | | |
| | | | | group: eyes | | |
| | | | | remained | | |
| | | | | unwashed | | |

target organism under defined conditions of exposure; IUPAC Compendium of Chemical Terminology, 2^{nd} Edition 1997, 65:2076 (1993), http://www.iupac.org/goldbook/N04208.pdf] exceeds 50,000 ppm Aquateric® daily in the diet. This represents an average dosage of 3,604 or 4,094 mg/kg/day for male and female rats, respectively, which is approximately 200 times the anticipated clinical topical dose of Aquateric® used as a microbicide.

In a chronic oral dosing experiment by Hodge (Hodge H, “The chronic toxicity of cellulose acetate phthalate in rats and dogs,” J. Pharmacol. Exp. Therapeutics, 80, 250-255 (1944)), four groups of 20 female rats each, were fed 0, 5, 20 and 30% CAP, ad libitum, for one year. The diet consisted of a Purina fox chow meal into which CAP was mixed and given ad libitum. The rats on high intake of CAP showed a reduction in growth rate, which increased with the dosage. No abnormalities were observed during autopsy. Histological examinations showed no consistent pathological changes. In general, no toxic effects of CAP have been found in rats.

Dog (Oral Study)

Three groups of 2 dogs each were fed 1, 4 or 16 gm of CAP daily for one year. The dogs remained in excellent health and condition throughout the experiment and no consistent pathological changes were discovered at autopsy. There was no evidence of toxic effects related to CAP in this study (Hodge H, “The chronic toxicity of cellulose acetate phthalate in rats and dogs,” J. Pharmacol. Exp. Therapeutics, 80, 250-255 (1944)). Feeding of CAP to rats or dogs for one year showed no evidence of target organ toxicity. In the subchronic study, rats received 0 (control), 5,000, 25,000, or 50,000 ppm (dose-range of 3600 to 4100 mg/kg/day) Aquateric® (containing 67% CAP) in the diet for 90 consecutive days. No mortality, clinical signs of toxicity or adverse toxicological effects on hematology or serum chemistry parameters, body weights, feed consumption, ophthalmological examinations, or histological evaluation of tissues were noted in any treatment group.

The following Table 3 is a summary of CAP repeat dose toxicity and carcinogenicity studies.

### Mutagenicity

Batt and Kotkoski (Batt K J, Kotkoskie L A, “An evaluation of genotoxicity tests with Aquateric aqueous enteric coating”, Internat. J. Toxicology, 18:117-122 (1999)) looked at the mutagenic potential of Micronized CAP in the Ames test, a mouse lymphoma mutation assay, and in a mouse micronucleus test. Results of all three tests were negative, suggesting that Micronized CAP is not mutagenic or genotoxic in this standard battery of tests (see the following Table 4).

Reproductive and Developmental Toxicity

Kotkoskie et al. (Kotkoskie L A, Freeman C, Palmieri M A, “Subchronic toxicity and developmental toxicity studies in rats with Aquateric® aqueous enteric coating,” Internat. J. Toxicology, 18:109-116 (1999)) examined developmental toxicity of Micronized CAP in rats. Groups of 25 pregnant Sprague-Dawley rats received 0, 5,000, 25,000, or 50,000 ppm of Micronized CAP in their diet ad libitum on days 6 through 15 of gestation. Upon sacrifice at day 20, no deaths and no significant differences in body weights or gravid uterine weights were observed. In addition there were no treatment-related, significant differences in Cesarean section parameters or observed gross lesions. Only one fetal malformation was noted (micrognathia), which was considered spurious and unrelated to treatment. There were no fetal external variations and no statistically significant differences in the fetal or litter incidences of visceral or skeletal variations.

Kotkoskie et al. (Kotkoskie L A, Freeman C, Palmieri M A, “Subchronic toxicity and developmental toxicity studies in rats with Aquateric® aqueous enteric coating,” Internat. J. Toxicology, 18:109-116 (1999)) also examined subchronic toxicity in 20 male Sprague-Dawley CD rats. Rats were administered Micronized CAP in diet at concentrations of 0, 5,000, 25,000, or 50,000 ppm for 90 consecutive days. Males receiving 50,000 ppm micronized CAP had decreased absolute testicular weights; however, relative testicular weights (testes to brain weight ratios) were unaffected. No histological alterations were present that correlated with the decrease in absolute testes weight.

The following Table 5 is a summary of CAP reproductive toxicity studies:

TABLE 3
Summary of CAP Repeat Dose Toxicity and Carcinogenicity Studies (from Literature)

| Species | | Group | Test | | Dose/ | | |
|---|---|---|---|---|---|---|---|
| (Strain) | Route | Size | Article | Formulation | Duration | Results | Reference |
| Rat | Oral | 20 F | CAP | CAP mixed | 0, 5, 20 30% | No toxic action of CAP was found in | Hodge, 1944 |
| (albino) | | | | with food | in food ad | rats. The rats on high intakes of CAP | |
| | | | | | libitum for 12 | showed a reduction in growth rate, | |
| | | | | | months. | which increased with the dosage. On | |
| | | | | | | autopsy, the rats were in good condition | |
| | | | | | | and no abnormalities were observed | |
| | | | | | | except that the average stomach weight | |
| | | | | | | tended to increase with higher doses of | |
| | | | | | | CAP. From histological examination, no | |
| | | | | | | consistent pathological changes were | |
| | | | | | | demonstrated. NOAEL > 30% in food | |
| Rat | Oral | 20 M + | 66-73 wt. % | Aquateric ® | 0, 5,000, | In a subchronic toxicity study no | Kotkoskie et |
| (Sprague- | | 20 F per | CAP in | mixed with | 25,000, | treatment-related deaths, hematological | al., 1999 |
| Dawley) | | group | Aquateric ® | food | 50,000 ppm | finding or clinical signs during the study. | |
| | | | | | in food for 90 | No toxicologically significant findings in | |
| | | | | | days | the clinical chemistry. No effect on body | |
| | | | | | | weights at 5,000 or 25,000 ppm | |
| | | | | | | Aquateric diet. Body weights were | |
| | | | | | | significantly reduced at 50,000 Aquateric | |
| | | | | | | diet investigations. NOAEL 25,000 ppm | |
| Dog | Oral | 6 animals | CAP | CAP mixed | 1, 4, 16 gm in | No evidence of any toxic effects of CAP. | Hodge, 1944 |
| (not | | | | with food | food/day for | The dogs remained in excellent health | |
| specified) | | | | | 12 months | and condition throughout the experiment | |
| | | | | | | and no consistent pathological changes | |
| | | | | | | were discovered at autopsy. NOAEL > | |
| | | | | | | 16 gm | |

TABLE 4
Summary of CAP Genotoxicity Studies (from Literature)

| Species | | Test | | | | |
|---|---|---|---|---|---|---|
| (Strain) | Route | Article | Formulation | Dose/Duration | Results | Reference |
| Mice | Oral | 66-73 wt. % | Aquateric ® | 7,200 mg/kg/ | Mouse Micronucleus Assay: During the mouse | Batt and |
| (CD-1) | | CAP in | | single dose | micronucleus assay all animals appeared normal after | Kotkoskie, |
| | | Aquateric ® | | | dosing and remained healthy until the end of the study. | 1999 |
| | | | | | Aquateric ® did not induce significant increases in | |
| | | | | | micronucleated polychromatic erythrocytes when | |
| | | | | | compared to the vehicle controls in either male or | |
| | | | | | female mice at any of the harvest times. NOAEL > | |
| | | | | | 7,200 mg/kg | |
| In Vitro | In | 66-73 wt. % | Aquateric ® | 0, 50, 167, 500, | Ames Test: Aquateric ® was not toxic to the Salmonella | Batt and |
| (Salmonella | Vitro | CAP in | solubilized in | 1,667, | tester strains (TA1535, TA1537, TA1538, TA98, | Kotkoskie, |
| typhimurium) | | Aquateric ® | dimethylsulfoxide | 5,000 μg/plate in | TA100). At the maximum dose, the test material did not | 1999 |
| | | | (DSMO) | 50 μl | cause an increase in the mean number of revertants | |
| | | | | DMSO/incubation at | compared to the solvent control with or without | |
| | | | | 37° C. for 48 h | metabolic activation by Aroclor-induced rat liver | |
| | | | | | microsomes in any of the tester strains. NOAEL > | |
| | | | | | 5,000 μg/plate in 50 μl DMSO | |
| In Vitro | In | 66-73 wt. % | Aquateric ® in | 116, 231, 750, | Mouse Lymphoma Assay in the absence of metabolic | Batt and |
| (L5178Y | Vitro | CAP in | deionized | 923, 1,500, 2,000 | activity: The test material was lethal at 2,500 μg/ml and | Kotkoskie, |
| TK+/− mouse | | Aquateric ® | water | μg/ml in | above. Treatment conditions were highly toxic at the | 1999 |
| lymphoma | | | | the absence of | 2,000 μg/ml dose with moderate to no toxicity at the | |
| cells) | | | | metabolic | lower concentrations. Mutant frequencies for the | |
| | | | | activation/incubation | treated cultures ranged from 31.3 × 10⁻⁶-55.0 × 10⁻⁶ and | |
| | | | | at 37° C. for | did not meet the criteria for a positive or mutagenic | |
| | | | | 4 hours with 2- | response. NOAEL > 1,500 μg/ml | |
| | | | | day recovery | | |
| | | | | and expression | | |
| | | | | period | | |
| In Vitro | In | 66-73 wt. % | Aquateric ® in | 116, 231, 554, 738, | Mouse Lymphoma Assay in the presence of metabolic | Batt and |
| (L5178Y | Vitro | CAP in | deionized | 923, 1,250 μg/ml in | activity: At 1,250 μg/ml, there was relative growth with | Kotkoskie, |
| TK+/− mouse | | Aquateric ® | water | the presence of | moderate to no toxicity at the remaining concentrations. | 1999 |
| lymphoma | | | | metabolic | All doses above 1,250 μg/ml were lethal to the cells. | |
| cells) | | | | activation/incubation | None of the cultures treated with the test material had | |
| | | | | at 37° C. for 4 hours | mutant frequencies which exceeded the minimum | |
| | | | | with 2-day recovery | criteria for a positive or mutagenic response. Mutant | |
| | | | | and expression | frequencies of the treated cultures ranged from | |
| | | | | period | 36.3 × 10⁻⁶-58.0 × 10⁻⁶. | |
| | | | | | NOAEL > 1,250 μg/ml | |

TABLE 5
Summary of CAP Reproductive Toxicity Studies (from Literature)

| Species | | | Test | | | | |
|---|---|---|---|---|---|---|---|
| (Strain) | Route | Group Size | Article | Formulation | Dose/Duration | Results | Reference |
| Rat | Oral | 25 F/group | 66-73 wt. % | Aquateric ® | 0, 5,000, | No treatment-related maternal or | Kotkoskie et |
| (Sprague- | | (pregnant) | CAP in | mixed with | 25,000, 50,000 ppm | developmental fetal effects were | al., 1999 |
| Dawley) | | | Aquateric ® | food | in food/ | observed. No fetal external | |
| | | | | | from day 6 | variations or visceral | |
| | | | | | through day 15 | malformations were noted in any | |
| | | | | | of gestation | fetus examined. NOAEL > | |
| | | | | | | 50,000 | |
| Rat | Oral | 20 M/group | 66-73 wt. % | Aquateric ® | 0, 5,000, | Males receiving 50,000 ppm | Kotkoskie et |
| (Sprague- | | | CAP in | mixed with | 25,000, 50,000 ppm | Aquateric ® had decreased | al., 1999 |
| Dawley) | | | Aquateric ® | food | in food/90 | absolute testicular weights; | |
| | | | | | days | however, relative testicular | |
| | | | | | | weights (testes to brain weight | |
| | | | | | | ratios) were unaffected. No | |
| | | | | | | histological alterations were | |
| | | | | | | present that correlated with the | |
| | | | | | | decrease in absolute testes | |
| | | | | | | weight. | |

Local and Photo-Sensitization

Guinea Pig (Dermal Study)

The manufacturer of Aquateric® CD-910 (containing 66-73 wt. % micronized Cellulose Acetate 1,2-Benzenedicarboxylate (Cellulose Acetate Phthalate: CAP)) (FMC Corporation) performed a skin sensitization study with subsequent induction treatment on Hartley guinea pigs. After three induction treatments one week apart to skin treated with Aquateric®, it was determined that Aquateric® was non-sensitizing when topically applied to Hartley guinea pigs. No responses were noted among test animals following either the induction or challenge application. No irritation was noted among any of the challenge control guinea pigs during challenge. Animals in the positive control group exhibited definite sensitizing reactions following the challenge application. The solubility of CAP is reasonably high at pH 7 and above. CAP is only minimally soluble at pH 6; below pH 6 the solubility further decreases. More detailed studies carried out by using a new newly developed sensitive method for the spectrophotometric determination of CAP (Neurath A R, Strick N, “Quantitation of cellulose acetate phthalate in biological fluids as a complex with ruthenium red,” Anal. Biochem, 288:102-04 (2001)) revealed that the solubility of CAP is approximately 7 μg/ml at pH 5.5 and further decreases with decreasing pH. The NOAEL from a 90-day study in rats (Kotkoskie L A, Freeman C, Palmieri M A, “Subchronic toxicity and developmental toxicity studies in rats with Aquateric® aqueous enteric coating,” Internat. J.

Toxicology, 18:109-116 (1999)) was approximately 2, 450 mg soluble CAP/kg/day. This safe dose of CAP is practically unachievable if an insoluble micronized form of CAP is used, provided that the environment is kept at pH levels≦5.5.

As disclosed in US 2007/0082035A1, experiments have demonstrated the surprisingly high buffering capacity at low pH of micronized CAP. Results disclosed in US 2007/0082035A1 indicate that >30 ml of blood per gram of CAP is required to bring the pH to levels at which the solubility of CAP starts to increase (to >7 μg/ml). The volume of menstrual fluid needed per gram of CAP would be much higher than that required for blood. 448 mg of CAP in the form of a water dispersible film (Neurath A R, Strick N, Li Y Y, “Water dispersible microbicidal cellulose acetate phthalate film,” BMC Infect. Dis., 3:27 (2003), http://www.biomedcentral.com/content/pdf/1471-2334-3-27.pdf; Neurath A R, Strick, Li Y Y, ‘Water dispersible film,” US Pat. Application Publication No. 2005/0070501 A1 (published Mar. 21, 2005)) was used in these experiments. Rabbit blood was used in these experiments which has a similar buffering capacity to human blood. The simulated menstrual fluid was prepared as described in Geshnizgani A M, Onderdonk A B, “Defined medium simulating genital tract secretions for growth of vaginal microflora,” J. Clin. Microbiol., 30:1323-132, (1992)).

CAP can safely be used in many physiological environments in which it is in micronized form. Due to its high buffering capacity, CAP will provide a low pH. This new finding is essential for the application of micronized CAP, in distinct forms and formulations, as an anti-infective/general hygiene product.

The safety of micronized CAP was further established as described in US 2002/0082035A1 as follows. A 14-day rabbit irritation study was conducted, in which 1 ml of formulations containing 130 mg of micronized CAP were applied daily vaginally to rabbits. These studies established that CAP at the concentrations and volumes used may be considered acceptable for human use. In contrast, treatment of rabbits with “CONCEPTROL” vaginal gel, a commercially available vaginal contraceptive product, resulted in vaginal irritation in all rabbits, that would be considered borderline or unacceptable for human use.

A gel formulation of micronized CAP (130 mg/g) was also applied vaginally to rhesus monkeys. Serum chemistries, vaginal biopsies, bacterial cultures and vaginal pH were determined to be within normal limits after dosing with CAP formulations. No obvious changes in peripheral CD4:CD8 cell ratios or levels of inflammatory cytokines/chemokines in plasma and vaginal fluids were detected. Colposcopy examinations determined that CAP formulations were not irritating (Ratterree M, et al., AIDS, 19, 1595 (2005)):

A method has been developed for delivering a freshly made, water-based CAP gel. This method uses a delivery system comprising an applicator with two compartments in which solid Aquateric® (which is a composition containing 66-73 wt. % CAP, polyoxyethylene-polyoxypropylene block copolymers and distilled acetylated monoglycerides) suspended in a thickened non-aqueous liquid (glycerin), (FMC Corporation, Philadelphia, Pa.) is separated from a water-based bioadhesive gel by a frangible seal. The final CAP containing gel is formed after breaking the seal followed by manual mixing of the powder component with the bioadhesive gel and then expelling the resulting gel mixture after breaking a further seal (see US 2007/0082035A1).

The use of a CAP as a microbicide is disclosed in the following: U.S. Pat. No. 5,985,313; U.S. Pat. No. 6,165,493; U.S. Pat. No. 6,462,030; U.S. Pat. No. 6,572,875; U.S. Pat. No. 6,596,297; and US 2005/0070501.

As indicated in the U.S. patents and U.S. patent publication identified in the preceding paragraph relating to CAP as a microbicide, micronized CAP binds HIV-1 virus particles, leading to HIV-1 gp41 6-helix bundle formation, virus inactivation and shedding of the gp120 envelope glycoproteins. This results in the rapid loss of infectivity. This has been demonstrated for a range of HIV clades and strains, including R5 strains (i.e., HIV strains which attach to the host cells' CCR5 chemokine receptors), the strains now considered to have the dominant role in sexual transmission of the virus.

CAP is also potent against other sexually transmitted pathogens, including HSV-2 (a virus responsible for genital herpes), Neisseria gonorrhoeae, Chlamydia trachomatis, Trichomonas vaginalis, Haemophilus ducreyi and Treponema pallidum. CAP also inactivates several bacteria associated with bacterial vaginosis (G. vaginalis, M. hominis, M. curtisii, P. corporis). CAP has no effect on Lactobacilli, bacteria that are natural components of the flora to be found in the healthy human vagina and that secrete lactic acid and hydrogen peroxide, which both offer some protection against sexually transmitted pathogens.

The above findings are derived from in vitro experiments and also from in vivo studies using animal models, including (a) the monkey model for genital simian immunodeficiency virus (SIV) infection, (b) the mouse model for genital herpesvirus infection and (c) the macaque model, using hybrid SIV/HIV-1 viruses (SHIV), for genital transmission of both subtypes X4 and R5.

### SUMMARY OF THE INVENTION

An object of the present invention is to provide a tablet which can be easily inserted into the vagina and, in the presence of vaginal fluid, is rapidly converted into a bioadhesive gel.

A further object of the present invention is to provide a microbicidal tablet for insertion into the vagina.

It is another object of the present invention to provide safe and relatively inexpensive methods, under the control of a woman, to prevent transmission of sexually transmitted diseases, such as human immunodeficiency virus, human cytomegalovirus, herpesvirus and bacterial vaginosis or an infection caused by Neisseria gonorrhoeae, Chlamydia trachomatis, Trichomonas vaginalis, Haemophilus ducreyi or Treponema pallidum.

It is a further object of the present invention to provide a method to treat or prevent bacterial vaginosis.

The above objects, as well as other objects and advantages, are achieved by the present invention.

The above-discussed formulation problems associated with CAP have now been overcome by the present inventors who have invented a vaginal tablet (solid dosage formulation) which can contain CAP.

A tablet of the present invention for insertion into a vagina including the following: 0.01 to 500 mg of at least one vaginal medication (active pharmaceutical ingredient (“API”)), such as an anti-infective agent, 100 to 500 mg of mannitol powder, 50 to 300 mg of inert microcrystalline cellulose, 10 to 80 mg of hydroxypropyl methylcellulose, 50 to 250 mg of glycerol and optionally 2 to 4 mg of at least one preservative which protects against microbial contamination and discourages the growth of Candida albicans (yeast) in the vagina.

A method for preventing the sexual transmission of HIV-1 or HIV-2 comprising vaginally administering to a human female before coitus the tablet described above, wherein the at least one vaginal medication is at least one microbicide, for example, CAP, a sodium salt of polynaphthalene sulfonic acid, an HIV replication inhibitor (such as an antiretroviral drug, for example, tenofovir (PMPA) or TMC-120); an HIV entry inhibitor targeting gp120 (such as CCR5 or CXCR4), an HIV adsorption inhibitor or an acid buffer.

A method for preventing the sexual transmission of HSV-1 comprising vaginally administering to a human female before coitus the tablet described above, wherein the at least one vaginal medication is selected from the group consisting of CAP, a sodium salt of PNSA and an acid buffer.

A method for preventing the sexual transmission of HSV-2 comprising vaginally administering to a human female before coitus the tablet described above, wherein the at least one vaginal medication is selected from the group consisting of CAP, a sodium salt of PNSA and an acid buffer.

A method for preventing the sexual transmission of human cytomegalovirus comprising vaginally administering to a human female before coitus the tablet described above, wherein the at least one vaginal medication is CAP.

A method for treating or preventing bacterial vaginosis comprising vaginally administering to a human female in need thereof the tablet described above, wherein the at least one vaginal medication is CAP.

A method for preventing the sexual transmission of an infection caused by Neisseria gonorrhoeae comprising vaginally administering to a human female before coitus the tablet described above, wherein the at least one vaginal medication is selected from the group consisting of CAP and a sodium salt of PNSA.

A method for preventing the sexual transmission of an infection caused by Chlamydia trachomatis comprising vaginally administering to a human female before coitus the table described above, wherein the at least one vaginal medication is selected from the group consisting of CAP and a sodium salt of PNSA.

A method for preventing the sexual transmission of an infection caused by Trichomonas vaginalis comprising vaginally administering to a human female before coitus the tablet described above, wherein the at least one vaginal medication is CAP.

A method for preventing the sexual transmission of an infection caused by Haemophilus ducreyi comprising vaginally administering to a human female before coitus the tablet described above, wherein the at least one vaginal medication is CAP.

A method for preventing the sexual transmission of an infection caused by Treponema pallidum comprising vaginally administering to a human female before coitus the tablet described above, wherein the at least one vaginal medication is CAP.

A method for preventing the sexual transmission of human papilloma virus (“HPV”) comprising vaginally administering to a human female before coitus the tablet described above, wherein the at least one vaginal medication is carrageenan.

A method for preventing conception comprising vaginally administering to a human female before coitus, the tablet described above, wherein the vaginal medication is a spermicide.

### BRIEF DESCRIPTION OF THE DRAWING

FIG. 1 depicts a dose-response curve which presents the results of tests on various dilutions of a gel derived from a CAP tablet. This shows that 1 ml of saline containing just 4 mg of CAP results in ca. 100% inactivation of HIV-1 BaL in 5 minutes. HIV-1 BaL is a R5 strain of HIV, the type with a dominant role in the virus's sexual transmission.

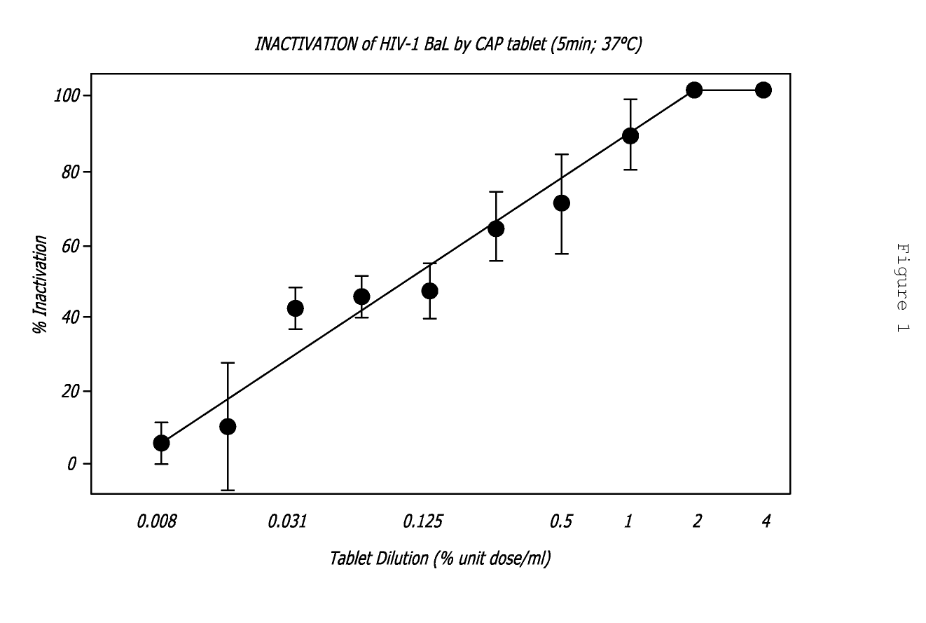

### DETAILED DESCRIPTION OF THE INVENTION

The present invention concerns a tablet of a size, shape and compactness so as to permit easy insertion into the vagina, either digitally or with an applicator. The tablet readily absorbs fluid to disintegrate rapidly in the limited volume of fluid generally present in the vagina, by virtue of its “wicking effect” that transports water to the tablet's interior, and by virtue of its high surface/volume ratio. The tablet contains a gelling agent that rapidly forms a smooth, stable bioadhesive gel when in contact with water.

The tablet of the present invention has all the required characteristics, disintegrating and forming a stable, smooth, bioadhesive, water-miscible, anti-infective gel within 2 to 3 minutes of placing it in approximately 2 ml fluid. The gel has an acidic pH and remains acidic (a pH of 3 to 5), even when mixed in vitro with a volume of semen typical of a human ejaculate.

It is considered that the tablet should be free of any significant local or systemic adverse effects, even after repeated use in the vagina.

The ingredients of the tablet for insertion into a vagina include 0.1 to 500 mg of at least one vaginal medication; 100 to 500 mg, preferably 200 to 400 mg of mannitol powder (which promotes rapid tablet disintegration); 50 to 300 mg, preferably 50 to 150 mg of inert microcrystalline cellulose; 10 to 80 mg, preferably 25 to 40 mg of hydroxypropyl methylcellulose; 50 to 250 mg, preferably 75 to 150 mg of glycerol; and optionally 2 to 4 mg of at least one preservative.

Preferred amounts of representative vaginal medications are as follows:

| | |
|---|---|
| CAP | 100 to 500 mg |
| Sodium salt of PNSA | 25 to 50 mg |
| Tenofovir | 30 to 60 mg |
| TMC-120 | 0.02 to 2 mg |
| Clotrimazole | 100 to 500 mg |
| Estradiol | 0.01 to 0.1 mg |

The at least one vaginal medication (active pharmaceutical ingredient (“API”)) is a medication that works on or through the vaginal mucosa. Examples of types of the at least one vaginal medication include at least one microbicide, at least one spermicide, at least one hormone, at least one antibiotic and at least one antifungal drug. The at least one vaginal medication may include a single vaginal medication or a combination of two or more vaginal medications of the same type or different types (for example, an acid buffer with PNSA or an antiretroviral drug other than CAP), with the proviso that there are no adverse interactions between two or more vaginal medications.

The microbicide is an anti-infective agent for preventing or treating infections caused by viruses, bacteria, fungi or protozoa.

Non-limiting examples of the microbicide include the following:
(1) CAP;
(2) At least one HIV replication inhibitor (antiretroviral replication inhibitor), for example, PMPA (tenofovir), TMC-120 (Dapivirine), MIV-150 (PETT) and UC-781;
(3) At least one HIV entry inhibitor targeting gp120, for example, cyanovirin-N, BMS-378806 and chimeric proteins with soluble CD4 (CD4-17b); targeting gp41, e.g., T20 (Fuzeon®, C52L or human monoclonal antibodies (e.g., 2F5 and 4E10); and targeting coreceptors CCR5 (e.g., Maraviroc, Aplaviroc, Vicriviroc, TAK779, NNY-RANTES and PSC-RANTES) and CXCR4 (e.g., AMD3100);
(4) At least one HIV adsorption inhibitor, for example, a sodium salt of polynaphthalene sulfonic acid (“PNSA”), carrageenan (CaraGuard™), naphthalene sulfonate polymer (PRO 2000) and dextrin-2-sulfate (Emmelle™); and
(5) At least one acid buffer, for example, Acidform™ and BufferGel™.

When CAP is utilized as the microbicide, a preferred form of CAP is micronized (particles of approximately 1 micron in size) cellulose acetate 1,2-benzenedicarboxylate in the form of a composition containing 66 to 73 weight % micronized cellulose acetate 1,2-benzenedicarboxylate with the remainder being polyoxyethylene-polyoxypropylene block copolymers and distilled acetylated monoglycerides.

Spermicides (contraceptives) for use in the tablets of the present invention include, but are not limited to, nonoxynol-9, benzalkonium chloride, octoxynol-9, cellulose sulfate, G31G (Savvy™) (a surfactant) and sodium dodecyl sulfate (“SDS”).

Non-limiting examples of hormones for use in the tablets of the present invention include estrogen (which can be used to rejuvenate the vaginal epithelium in older women) and progestagen.

As an antifungal and antibacterial drug, an imidazole drug, such as clotrimazole (which can be used against vaginal candidiasis (thrush)), econazole, isoconazole, enetronidazole (Flagyl) and miconazole, can be used in the tablets of the present invention.

The mannitol powder contributes to the tablet's physical stability and acts as a “wicking agent,” readily absorbing water and transporting it to the tablet's interior. The mannitol powder should preferably have a particle size not exceeding 150 microns.

The inert microcrystalline cellulose serves to form a thixotropic gel which helps to provide an effective suspension when micronized CAP is employed as the at least one vaginal medication. The inert microcrystalline cellulose has fine particles (i.e., not “gritty”), so as not to cause discomfort either to the female user of the tablet or to her male partner. The inert microcrystalline cellulose has a particle size of preferably 20 to 180 microns.

The hydroxypropyl methylcellulose (HPMC) serves as a rapidly soluble gelling agent to ensure that the resulting gel has the necessary viscosity, rheological and bioadhesive properties. A preferred viscosity of the HPMC is 3000 to 4000 cps.

Glycerol provides the hyper-osmolarity needed to withdraw fluid (transudate) through the vaginal epithelium into the vaginal lumen to assist in the tablet's disintegration.

The purpose of the at least one preservative is to guard against the possibility of vaginal candidiasis (yeast infection) and to prevent potential microbial contamination during production or storage of tablets under suboptimal conditions.

Depending on the at least one vaginal medication included in the tablet, e.g., CAP, which has a very slight acetic acid smell, a non-allergenic fragrance (e.g., vanilla, jasmine or rose) may be used to conceal the odor of the vaginal medication.

The thickness and overall shape of the tablet is determined to provide for optimal insertion and dispersion. The tablet should have a sufficiently large surface/volume ratio to optimize the uptake of vaginal fluid. The shape is preferably either (i) a thin rectangle with half-round ends or (ii) a thin oval. The ends and sides should preferably be rounded; preferably the tablet should not have any sharp edges.

For a 1 g tablet of the composition described herein, suitable dimensions are as follows:

length: 20 to 50 mm, preferably 26 to 32 mm, more preferably 30 mm
width: 6 to 16 mm, preferably 8 to 12 mm, more preferably 10 mm
thickness 2 to 10 mm, preferably 3 to 4 mm, more preferably 3 mm.

The tablet has a weight of approximately 400 mg to 2000 mg, preferably 400 mg to 1700 mg and more preferably 400 mg to 1200 mg.

The tablet described hereinabove and which comprises at least one microbicide (for example, CAP, an HIV replication inhibitor, an HIV entry inhibitor targeting gp120, an HIV adsorption inhibitor (such as a sodium salt of PNSA) or an acid buffer) as the vaginal medication, can be vaginally administered to prevent the sexual transmission of HIV-1 or HIV-2.

The tablet described hereinabove and which comprises CAP, a sodium salt of PNSA or an acid buffer as the vaginal medication, can be vaginally administered to prevent the sexual transmission of HSV-1 or HSV-2.

The tablet described hereinabove and which comprises CAP as the vaginal medication can be vaginally administered to treat or prevent bacterial vaginosis caused by a microorganism selected from the group consisting of Gardnella vaginalis, Mycoplasma hominis, Mycoplasma capricolum, Mobiluncus curtisii and Prevotella corporis.

The tablet described hereinabove and which comprises CAP or a sodium salt of PNSA as the vaginal medication can be vaginally administered to prevent the sexual transmission of human cytomegalovirus or an infection caused by a microorganism selected from the group consisting of Trichomonas vaginalis, Haemophilus ducreyi (which causes chancroid), Treponema pallidum, Chlamydia trachomatis and Neisseria gonorrhoeae.

The tablet described hereinabove and which comprises carrageenan as the vaginal medication can be vaginally administered to prevent human papilloma virus.

The tablet described hereinabove and which comprises a spermicide can be vaginally administered to prevent conception.

To make the tablet, the tableting process is arranged so that the core of the tablet is compressed very lightly (and is therefore rapidly dispersible), while the thin outer layer will be compressed to a greater degree to provide a physically robust tablet. This is achievable with standard equipment.

It is considered that a CAP tablet according to the present invention will have the following advantages:

(i) will reduce the risk of sexual transmission of the above-described organisms;

(ii) will be beneficial in the treatment of bacterial vaginosis; and

(iii) will be useful as a regular component of vaginal hygiene procedures.

Other advantages of microbicide tablets according to the present invention include the following:
Water-free, good stability, long shelf-life.
Ease and economy of manufacture, using fairly standard tableting technology, therefore far easier to achieve the necessary high global production rates than is the case with pre-prepared gels in pre-filled plastic applicators, requiring specialized technology.
Ease and economy of packaging: shrink-wrapped or blister-packed in plastic or aluminum; no gel-handling, or filling and sealing expensive plastic applicators/syringes.
Small bulk, therefore economic shipping, warehousing and local distribution.
Ease of storage and use by end-users, with only the wrapping to dispose of afterwards.
Low cost to consumers compared to gel/applicator microbicides.
Evidence that a vaginal tablet inserted by one's finger is likely to be more acceptable to many women (e.g., evidence from India and Sub-Saharan Africa), than a gel inserted by means of an applicator/syringe.
Long-held view of microbicide experts that a diversity of formats is needed to appeal to different user situations and different cultural groups.
Regulatory advantages, as FDA, EMEA and developing country authorities are used to vaginal tablets for therapeutic use.
Can be used for single agents or combinations, e.g., CAP plus a nucleotide reverse transcriptase inhibitor (NRTI) or non-nucleotide reverse transcriptase inhibitor (NNRTI) or other antiretroviral drug.

### EXAMPLES

### Example 1

### CAP Vaginal Tablet

Tablet weight: 1 g

Aquateric® 34.3 wt. % 343 mg (containing 241 mg CAP)

Aquateric is a commercial micronized product containing approximately 67 wt. % CAP (the active pharmaceutical ingredient of the vaginal tablets), such as from the FMC Corporation, Philadelphia, Pa. The remainder comprises a polyoxyethylene-polyoxypropylene block co-polymer and distilled acetylated monoglycerides.

Mannogem 30.9 wt. % 309 mg

This is a mannitol powder produced by SPI Polyols, Inc., New Castle, Del.

Avicel 14.3 wt. % 143 mg

Avicel is inert microcrystalline cellulose. Avicel Type PH-105 having a particle size of approximately 20 microns, is obtained from FMC BioPolymer, 1735 Market Street, Philadelphia, Pa. 19103, USA or Avenue Louise 480—B9, 1050 Brussels, Belgium.

Hydroxypropyl methylcellulose (HPMC) 3.4 wt. % 34 mg

It is preferred to use Metolose, Grade 90SH-4000SR, having a viscosity of 4000 cps, obtained from Shin-Etsu Chemical Co. Ltd., 6-1 Ohtemachi, 2-chome, Chiyoda-ku, Tokyo, Japan.

Glycerol 17.1 wt. % 171 mg

Preservatives:

| | | |
|---|---|---|
| Sodium benzoate | 0.1 wt. % | 1 mg |
| Methyl paraben sodium | 0.2 wt. % | 2 mg |
| Propyl paraben sodium | 0.03 wt. % | 0.3 mg |

The above combination including Mannogem, Avicel and glycerol results in a tablet which is converted into a bioadhesive gel and is rapidly penetrated by aqueous media, accelerating its disintegration.

All ingredients are effectively and uniformly mixed prior to pressing into tablets.

The tablet can be made by the following procedure:

The solid powder ingredients are thoroughly mixed in a beaker by hand, then the glycerol is added and thoroughly mixed in, again by hand. Weighed amounts of the mixture (either 1 gram or 0.4 grams according to the Examples set forth herein) are placed in a hand-operated tablet (pellet) press.

The tablet was prepared by using a pellet press with a punch and die set (Model No. 2811) purchased from Parr Instrument Company at 211 Fifty Third Street, Moline, Ill. 61265. The mixture containing the ingredients for making the tablet was filled into the die with ½″ diameter and 1″ height. The lever was pushed down by hand with proper pressure so that the punch entered into the die about ½″. Then the lever was raised to its top position to allow the finished tablet to be removed from the die.

### Example 2

### CAP Vaginal Tablet

The following tablet was made following the procedure and using the same ingredients as in Example 1.

Tablet weight: 1 g

| | |
|---|---|
| Aquateric ® | 30 wt % |
| Mannogem | 41 wt % |
| Avicel | 10 wt % |
| Hydroxypropyl methylcellulose | 4 wt % |
| Glycerol | 15 wt % |
| Sodium benzoate | 0.1 wt % |
| Methyl paraben sodium | 0.2 wt % |
| Propyl paraben sodium | 0.03 wt % |
| Fragrance (optional) | |

### Example 3

### Vaginal Tablet Containing the Sodium Salt of Polynaphthalene Sulfonic Acid

With minor modifications, the above approach can be used to formulate other microbicides as rapidly dispersible, fast-gelling vaginal tablets. For example, a sodium salt of polynaphthalene sulfonic acid (PNSA) can be used instead of CAP. PNSA in the form of PRO 2000 gel is currently the subject of two large-scale effectiveness trials (sponsored respectively by the US National Institutes of Health and the UK Medical Research Council) in communities at high-risk of HIV. An example of a tablet formulation utilizing a sodium salt of PNSA is as follows (tablet weight 0.4 g):

| | | |
|---|---|---|
| Sodium salt of polynaphthalene sulfonic acid | 12.5 wt % | 50 mg |
| Mannogem | 47.5 wt % | 190 mg |
| Avicel | 14.2 wt % | 57 mg |
| Hydroxypropyl methylcellulose (“HPMC”) | 6.2 wt % | 25 mg |
| Glycerol | 18.7 wt % | 75 mg |
| Sodium benzoate | 0.2 wt % | 1 mg |
| Methyl paraben sodium | 0.4 wt % | 2 mg |
| Propyl paraben sodium | 0.06 wt % | 0.3 mg |
| Fragrance (optional) | | |

### Example 4

### Vaginal Tablet Containing Tenofovir

The following vaginal tablet can be made according to the procedure set forth in Example 1 and having the following composition:

| | |
|---|---|
| Tenofovir (a nucleotide reverse | 30 mg |
| transcriptase inhibitor) | |
| Mannitol | 190 mg |
| Microcrystalline cellulose | 57 mg |
| HPMC | 25 mg |
| Glycerol | 75 mg |
| Sodium benzoate | 1 mg |
| Methyl paraben sodium | 2 mg |
| Propyl paraben sodium | 0.3 mg |
| Fragrance (optional) | |

### Example 5

### Vaginal Tablet Containing Clotrimazole

The following vaginal tablet can be made according to the procedure set forth in Example 1 and having the following composition:

| | |
|---|---|
| Clotrimazole | 300 mg |
| Mannitol | 410 mg |
| Microcrystalline cellulose | 100 mg |
| HPMC | 40 mg |
| Glycerol | 150 mg |

### Example 6

### Vaginal Tablet Containing Estradiol

The following vaginal tablet can be made according to the procedure set forth in Example 1 and having the following composition:

| | |
|---|---|
| Estradiol | 0.025 mg |
| Mannitol | 220 mg |
| Microcrystalline cellulose | 70 mg |
| HPMC | 27 mg |
| Glycerol | 80 mg |
| Sodium benzoate | 1 mg |
| Methyl paraben sodium | 2 mg |
| Propyl paraben sodium | 0.3 mg |

### Example 7

### Anti-HIV Activity of CAP in the Vaginal Tablets

The inhibitory activity of CAP in the vaginal tablets on infection by primary HIV-1 isolates in CEM×174 5.25M7 cells was determined as previously described (Lu et al., AIDS Res. Hum. Retroviruses 22: 411-418, 2006). 30 mg of the CAP tablet of Example 2 was suspended in 1 ml of PBS, and then diluted in RPMI-1640 medium to keep the CAP concentration at 4 mg/ml. Fifty μl of serially four-fold diluted CAP-containing samples were incubated with an equal volume of a primary HIV-1 isolate (obtained from the NIH AIDS Research and Reference Reagent Program) at 0.01 multiplicity of infection (MOI) at 37° C. for 30 minutes, followed by addition of 100 μl CEM×174 5.25 M7 cells (5×10⁵/ml). After incubation at 37° C. overnight, the culture supernatants were replaced with fresh medium. On day 3 post infection, the cells were harvested and lysed for analysis of luciferase activity using a luciferase assay kit (Promega, Madison, Wis.) and a luminometer (Model: Ultra 386, Tecan, Durham, N.C.)' according to the manufacturer's instruction. The percent inhibition of luciferase activity and the IC₅₀ and IC₉₀ values were calculated as described before (Lu et al., AIDS Res. Hum. Retroviruses 22: 411-418, 2006). As shown in Table 6, CAP in the vaginal tablets effectively inhibited infection by primary HIV-1 isolates with distinct genotypes, including subtypes A, B, C, E and EA and biotypes (R5, X4, and X4R5), suggesting that CAP in the vaginal tablets retains its potent and broad anti-HIV-1 activity.

TABLE 6
CAP in the Vaginal Tablets Inhibited
Infection by Primary HIV-1 Isolates

| Primary | Subtype, | IC₅₀ (μg/ml) | IC₉₀ (μg/ml) |
|---|---|---|---|
| HIV-1 isolate | coreceptor usage | (mean ± SD) | (mean ± SD) |
| 92RW008 | A, R5 | 37.24 ± 2.65 | 145.90 ± 10.03 |
| 92US657 | B, R5 | 41.53 ± 4.09 | 258.55 ± 5.29 |
| 93IN101 | C, R5 | 11.88 ± 0.36 | 216.68 ± 11.06 |
| 92TH009 | E, R5 | 64.15 ± 2.09 | 172.31 ± 4.28 |
| 93TH051 | E, X4R5 | 24.71 ± 12.94 | 85.33 ± 29.41 |
| CMU02 | EA, X4 | 2.07 ± 1.11 | 12.08 ± 4.14 |

The samples were tested in triplicate.

### Example 8

### Stability of CAP in the Vaginal Tablets

An accelerated stability study was carried out to determine the stability of CAP in the vaginal tablets of Example 2 stored at different temperatures (4° C., room temperature, 30° C. and 40° C., respectively) for 1 to 13 weeks. High Performance Liquid Chromatography (HPLC) for CAP was performed in a Waters 600E multisolvent delivery system with a Waters 996 photodiode array detector (detection at 254 nm) and a VYDAC 301 VHP 575 column equilibrated with 20 mM borate, pH 8.5 (buffer A). Samples were diluted in buffer A, followed by adjustment of pH to 8.5 and centrifugation at 3000×g for 5 minutes. The supernatants (20 μl) were applied to the column, which was subsequently eluted with a linear gradient (buffer A buffer B: 1 M NaCl in buffer A) at a flow rate of 1 ml/minute. The retention time for free phthalic acid released from CAP as a result of hydrolysis was less than 2 minutes and for CAP was between 6 and 10 minutes, respectively. As shown in Table 7, more than 85% of CAP is detectable in the vaginal tablets stored at 4° C., room temperature (“RT”) and 30° C. for 3 months, while about 30% of CAP was degraded when the tablets were stored at 40° C. for more than 2 months. These results suggest that CAP is stable in the vaginal tablets stored at regular temperatures in most residential areas in the world, except those in tropical regions.

TABLE 7
Stability of CAP in the Vaginal Tablets

| Intervals | 4° C. | RT | 30° C. | 40° C. |
|---|---|---|---|---|
| Week 1 | 93.87 | 91.24 | 90.32 | 88.38 |
| Week 2 | 90.77 | 91.59 | 89.46 | 86.31 |
| Week 3 | 93.24 | 89.95 | 88.22 | 84.13 |
| Week 4 | 95.42 | 90.09 | 88.59 | 82.42 |
| Week 5 | 96.05 | 88.04 | 87.69 | 80.19 |
| Week 6 | 95.92 | 88.95 | 88.06 | 79.08 |
| Week 7 | 90.83 | 88.78 | 86.14 | 77.89 |
| Week 8 | 95.76 | 89.07 | 86.46 | 75.53 |
| Week 9 | 95.4 | 88.72 | 85.79 | 71.46 |
| Week 10 | 92.45 | 87.52 | 86.61 | 73.19 |
| Week 11 | 93.57 | 86.87 | 83.04 | 69.06 |
| Week 12 | 94.3 | 89.32 | 84.11 | 71.86 |
| Week 13 | 94.45 | 88.34 | 85.79 | 68.61 |

It will be appreciated that the instant specification is set forth by way of illustration and not limitation, and that various modifications and changes may be made without departing from the spirit and scope of the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A tablet for insertion into a vagina comprising:
0.01 to 500 mg of at least one vaginal medication,
100 to 500 mg of mannitol powder,
50 to 300 mg of inert microcrystalline cellulose,
10 to 80 mg of hydroxypropyl methylcellulose and
50 to 250 mg of glycerol.

2. The tablet according to claim 1, which further comprises 2 to 4 mg of at least one preservative which discourages the growth of yeast in the vagina.

3. The tablet according to claim 1, wherein the at least one vaginal medication is selected from the group consisting of at least one microbicide, at least one spermicide, at least one hormone, at least one antibiotic and at least one antifungal drug.

4. The tablet according to claim 1, wherein the at least one vaginal medication is a microbicide selected from the group consisting of CAP, a sodium salt of PNSA, at least one HIV replication inhibitor, at least one HIV entry inhibitor and at least one acid buffer.

5. The tablet according to claim 1, wherein the at least one vaginal medication comprises micronized cellulose acetate 1,2-benzenedicarboxylate.

6. The tablet according to claim 5, wherein the at least one vaginal medication comprises 66 to 73 weight % of the micronized cellulose acetate 1,2-benzenedicarboxylate, with the remainder being polyoxyethylene-polyoxypropylene block co-polymer and distilled acetylated monoglycerides.

7. The tablet according to claim 2, wherein the at least one preservative is selected from the group consisting of sodium benzoate, methyl paraben sodium and propyl paraben sodium.

8. The tablet according to claim 1, which further comprises a fragrance.

9. The tablet according to claim 1, wherein the tablet has a length of 20 to 50 mm, a width of 6 to 16 mm and a thickness of 2 to 10 mm.

10. The tablet according to claim 1, wherein the tablet has a length of 26 to 32 mm, a width of 8 to 12 mm and a thickness of 3 to 4 mm.

11. The tablet according to claim 1, wherein the tablet is in a shape of a thin rectangle with half-round ends.

12. The tablet according to claim 1, wherein the tablet is in a shape of a thin oval.

13. The tablet according to claim 1, wherein the at least one vaginal medication is a sodium salt of polynaphthalene sulfonic acid.

14. A method for preventing the sexual transmission of HIV-1 or HIV-2 comprising vaginally administering to a human female before coitus the tablet according to claim 1, wherein the at least one vaginal medication is at least one microbicide.

15. (canceled)

16. A method for preventing the sexual transmission of HSV-1 comprising vaginally administering to a human female before coitus the tablet according to claim 1, wherein the at least one vaginal medication is selected from the group consisting of CAP, a sodium salt of PNSA and an acid buffer.

17. A method for preventing the sexual transmission of HSV-2 comprising vaginally administering to a human female before coitus the tablet according to claim 1, wherein the at least one vaginal medication is selected from the group consisting of CAP, a sodium salt of PNSA and an acid buffer.

18. A method for preventing the sexual transmission of human cytomegalovirus comprising vaginally administering to a human female before coitus the tablet according to claim 1, wherein the at least one vaginal medication is CAP.

19. A method for treating or preventing bacterial vaginosis comprising vaginally administering to a human female in need thereof the tablet according to claim 1, wherein the at least one vaginal medication is CAP.

20. A method for preventing the sexual transmission of an infection caused by Neisseria gonorrhoeae comprising vaginally administering to a human female before coitus the tablet according to claim 1, wherein the at least one vaginal medication is selected from the group consisting of CAP and a sodium salt of PNSA.

21. A method for preventing the sexual transmission of an infection caused by Chlamydia trachomatis comprising vaginally administering to a human female before coitus the tablet according to claim 1, wherein the at least one vaginal medication is selected from the group consisting of CAP and a sodium salt of PNSA.

22. A method for preventing the sexual transmission of an infection caused by Trichomonas vaginalis comprising vaginally administering to a human female before coitus the tablet according to claim 1, wherein the at least one vaginal medication is CAP.

23. A method for preventing the sexual transmission of an infection caused by Haemophilus ducreyi comprising vaginally administering to a human female before coitus the tablet according to claim 1, wherein the at least one vaginal medication is CAP.

24. A method for preventing the sexual transmission of an infection caused by Treponema pallidum comprising vaginally administering to a human female before coitus the tablet according to claim 1, wherein the at least one vaginal medication is CAP.

25. A method for preventing the sexual transmission of human papilloma virus comprising vaginally administering to a human female before coitus the tablet according to claim 1, wherein the at least one vaginal medication is carrageenan.

26. A method for preventing conception comprising vaginally administering to a human female before coitus, the tablet according to claim 1, wherein the vaginal medication is a spermicide.
